(19) United States
(12) Patent Application Publication
(10) Pub. No.: US 2022/0270721 A1
(43) Pub. Date: Aug. 25, 2022

(54) TEXT PROCESSING METHOD AND APPARATUS

(71) Applicant: CANON MEDICAL SYSTEMS CORPORATION, Otawara-shi (JP)

(72) Inventors: Patrick SCHREMPF, Edinburgh (GB); Alison O'NEIL, Edinburgh (GB); Hannah WATSON, Edinburgh (GB)

(73) Assignee: CANON MEDICAL SYSTEMS CORPORATION, Otawara-shi (JP)

(21) Appl. No.: 17/450,339

(22) Filed: Oct. 8, 2021

Related U.S. Application Data
(60) Provisional application No. 63/152,461, filed on Feb. 23, 2021.

(51) Int. Cl. G16H 10/60; G06F 40/186

(52) U.S. Cl. CPC G16H 10/60; G06F 40/186

(57) ABSTRACT

A medical information processing apparatus is for processing medical text data using a medical text processing model. The apparatus comprising processing circuitry configured to: receive a portion of medical text data; determine a template corresponding to the portion of medical text data, a classification label associated with the template, and a first medical term included in the portion of medical text data; specify a second medical term which is different from the first medical term, wherein the second medical term is related to the first medical term; and generate synthetic text data based on the second term by inserting the second medical term into the template.

### FIELD

Embodiments described herein relate generally to a method and apparatus for processing text data, for example for training models to process text data using templates and/or synthesized text data.

### BACKGROUND

It is known to apply machine learning models to process text data. In some applications, the language used in the text data to be analyzed is specialized and domain-specific. For example, medicine has a large specialized vocabulary and may often express language in a way that is different from how that language is used in a more general domain.

It is known to perform natural language processing (NLP), in which free text or unstructured text is processed to obtain desired information. For example, in a medical context, the text to be analyzed may be a clinician's text note. The clinical text note may be stored within an Electronic Medical Record. The clinical text note may be a free-text radiology report. The text may be analyzed to obtain information about, for example, a medical condition or a type of treatment.

The free-text radiology report may be associated with one or more medical images. It is known to perform analysis of medical images to obtain information about, for example, anatomy or pathology. Analysis may be performed manually by a radiologist. Analysis may be performed automatically, for example by a trained image analysis model.

Training medical image analysis models requires large amounts of expertly annotated imaging data which is time-consuming and expensive to obtain. Fortunately, images are often accompanied by free-text radiology reports which are a rich source of information, containing the radiologist's summary of what they see (findings) and what they consequently diagnose (clinical impressions). A finding is what the radiologist sees in the image. An example of a finding is hyperdensity. An impression is what the radiologist diagnoses based on the findings. An example of an impression is haemorrhage.

Recent approaches to creating large imaging datasets have involved mining these reports to automatically obtain image-level labels. Image-level labels can then be used to train anomaly detection algorithms, as done for instance in the RSNA haemorrhage detection challenge (Radiological Society of North America. RSNA Intracranial Hemorrhage Detection, Kaggle challenge https://www.kaggle.com/c/rsna-intracranial-hemorrhage-detection/overview) and the CheXpert challenge for automated chest X-Ray interpretation (Irvin, J.; Rajpurkar, P.; Ko, M.; Yu, Y.; Ciurea-Ilcus, S.; Chute, C.; Marklund, H.; Haghgoo, B.; Ball, R.; Shpanskaya, K.; others. CheXpert: A large chest radiograph dataset with uncertainty labels and expert comparison. Proceedings of the AAAI Conference on Artificial Intelligence, 2019, Vol. 33, pp. 590-597).

However, extracting labels from text can be challenging because the language in radiology reports is diverse, domain-specific, and often non-committal where phenomena are indistinct or ambiguous. Therefore, the task of reading the radiology report and assigning labels is not trivial and requires a certain degree of medical knowledge on the part of a human annotator. Sometimes even medical experts disagree on which label should be extracted from a given text.

Natural language processing may be performed using deep learning methods, for example using a neural network.

Deep learning is becoming a dominant technique for natural language processing tasks, for example information retrieval, entity recognition, document classification, abstractive summarization and natural language generation.

Deep learning algorithms may be trained on annotated clinical text sentences or documents.

Deep learning algorithms may be trained for extraction of imaging entities from radiology reports. Deep learning algorithms may be trained for ICD (International Statistical Classification of Diseases and Related Health Problems) coding of discharge summaries.

Supervised algorithms may be trained on annotated clinical text sentences or documents. The annotated clinical text may be data that is annotated by an expert, for example a clinician. The annotations may include ground truth classifications.

An example of training on annotated clinical text using a standard machine learning system is illustrated schematically in FIG. 1.

An annotated clinical text corpus 1 is received. The annotated clinical text corpus may comprise, for example, a plurality of annotated radiology reports. Annotations to the radiology reports may comprise, for example, classification labels.

The annotated clinical text corpus 1 is used as training input for training a deep learning model 2. The deep learning model 2 is trained to provide model outputs for each of a plurality of classes. The model outputs may include, for each class, document or sentence-level probabilities. The model outputs may include, for each class, a word-level attention weighting.

In one example, the deep learning model 2 is trained to obtain predictions for a certain set of labels for each sentence of the radiology report and/or for the radiology report as a whole. Each label relates to a corresponding finding or impression. For example, labels may include haemorrhage and tumour. The deep learning model 2 may be trained to classify each sentence or report to say whether each of haemorrhage and tumour is present.

For each sentence, each of the labels is classified in one of a plurality of certainty classes. The certainty classes include positive, uncertain and negative. A classification in the positive certainty class is made when the model determines from the sentence that the finding or impression that is represented by the label is present. A classification in the negative certainty class is made when the model determines from the sentence that the finding or impression that is represented by the label is not present. A classification in the uncertain certainty class is made when the model determines from the sentence that the presence of the finding or impression is uncertain. For example, the sentence may suggest that the finding or impression may be present without providing a strong enough indication to be classified as positive.

In FIG. 1, the deep learning model 2 classifies three terms 4, 5, 6 in a text document 3 as positive, negative or uncertain.

The text document 3 comprises the text:
“Clinical History
72 yo man. Known uncontrolled hypertension, previous TIA, type 2 diabetes mellitus. Presented visual symptoms.
CT Head:
Axial non-contrast.
The lateral ventricles are minimally asymmetric as previously which may be congenital or secondary to the left basal ganglia haemorrhage. No immediate threat from swelling.”

The deep learning model 2 classifies a first term 4 in the text 3, which is ‘congenital’. The deep learning model 2 classifies the first term 4 as uncertain.

The deep learning model 2 classifies a second term 5 in the text 3, which is ‘haemorrhage’. The deep learning model 2 classifies the second term 5 as positive.

The deep learning model 2 classifies a third term 6 in the text 3, which is ‘swelling’. In the example of FIG. 1, the deep learning model 3 classifies the third term 6 as negative. The model predicts that ‘swelling’ is negative (not present) but it should be labelled positive. In this case, the model may have used the presence of the word ‘No’ in the last sentence of the text to predict a negative classification. The classification is incorrect because the word ‘No’ in the last sentence of the text relates to the threat, rather than to the swelling.

The process of FIG. 1 may be described as data-driven learning. A model is trained on a training data set, for example a set of radiology reports that has been labelled by an expert. The model learns from the data and the labels.

If pure data-driven learning is relied on, it is found that the model sometimes omits to learn critical features. The model may sometimes learn the correct answer via simple heuristics (for example, presence of the word “not”) rather than comprehensive reasoning. See, for example, McCoy, T., Pavlick, E. and Linzen, T., 2019, July. Right for the Wrong Reasons: Diagnosing Syntactic Heuristics in Natural Language Inference. In Proceedings of the 57th Annual Meeting of the Association for Computational Linguistics (pp. 3428-3448). This may be particularly problematic when there are few training instances of difficult variants.

For instance, it may be difficult to train the model to provide predictions for rare or new classes. We may have no examples, or only a few examples, of a given class. For example, there may be no or few examples of rare pathologies in a training set. Data-driven models may have difficulty in teaching a model about unseen classes.

There may be ambiguous wording that requires expert consensus to interpret. Some wording may require human judgement to interpret. For example, a report may include wording around uncertainty such as “A tumour is suspected” or “Hyper density could be haemorrhage or calcification”. Data-driven models may have difficulty in teaching a model about rules concerning ambiguous wording.

A model may require a contextual understanding, for example temporal or anatomical understanding. Sometimes the context of a given term is important. For example, a radiology report may include the text “worth MRI to confirm or refute a partial left MCA territory infarct”. In this example, the infarct is not mentioned with respect to the current image. The infarct may not be in the image. For example, the infarct may be looked for in a future scan.

In another example, a radiology report may include the text “Ongoing maturation and reducing density of the previous right hemisphere intraparenchymal and subarachnoid haemorrhage”. The presence of the term ‘haemorrhage’ within the sentence provides the context that allows ‘reducing density’ to be interpreted as ‘reducing hyperdensity’.

In another example, a sentence may read ‘Ongoing maturation and reducing density compared to previously’. In this case, context outside the sentence is important to confirm that there is a hyperdensity rather than a hypodensity present. For example, the context of haemorrhage may be provided by another nearby sentence. Data-driven models may have difficulty in teaching a model about the importance of context.

In some circumstances, labelling rules may be task-specific. For example, “there is low attenuation within the left cerebellum and probably the posterior inferior cerebellar artery territory” contains both a positive and an uncertain mention of hypodensity. In a sentence classification task, the sentence is labelled as a positive classification as this takes precedence over the uncertain mention. A data-driven model may have difficulty in learning task-specific rules. In some examples, a model may classify any sentence with a positive mention as a positive classification at all times, which may not be appropriate to all contexts.

Data synthesis and augmentation are known techniques for increasing an amount of training data for deep learning models. Various approaches can be used to generate the synthetic data, including:

a) Simple text manipulation, such as deleting words from or adding words to a text. For example, a random word may be removed or inserted, or a word may be duplicated.

b) Trained sequence to sequence (Seq2Seq) models which are capable of generating text.

c) Generative adversarial models. In the imaging domain, generative adversarial networks (GANs) have been very successful recently. However, these have not been found to be so successful in natural language processing.

Examples of some text augmentation techniques used to create synthetic data for abstractive summarization include paraphrasing; sentence negation; pronoun swap; entity swap; number swap; and noise injection.

Paraphrasing is usually achieved by back-translation using neural machine translation models and has been used widely. Dividing text in the training corpus to provide additional examples has also been shown to improve classification performance. These approaches introduce greater diversity to the training set to reduce overfitting. However, it is often found that deep learning models suffer from the opposite problem of underfitting in situations where syntactic heuristics can be learned in lieu of the textual nuances that are critical to distinguish less common cases and this is more likely with a small training dataset, which is often the case in the medical field.

### SUMMARY

In a first aspect, there is provided a medical information processing apparatus for processing medical text data using a medical text processing model, the apparatus comprising processing circuitry configured to: receive a portion of medical text data; determine a template corresponding to the portion of medical text data, a classification label associated with the template, and a first medical term included in the portion of medical text data; specify a second medical term which is different from the first medical term, wherein the second medical term is related to the first medical term; and generate synthetic text data based on the second term by inserting the second medical term into the template

The processing circuitry may be further configured to train the medical text processing model using the synthetic text data and the classification label.

The synthetic text data may be generated by inserting the second term into the template in a position corresponding to a position of the first medical term in the portion of medical text data.

The processing circuitry may be further configured to: determine at least one of the first medical term, the second medical term, the template, the label for each of a plurality of predetermined data distribution groups.

The second medical term may be a synonym of the first medical term.

The processing circuitry may be further configured to determine the synonym of the first medical term using at least one of: a database, a knowledge base, a knowledge graph, an ontology.

The portion of medical text data may be a sentence or part of a sentence.

The classification label may comprise a classification of the portion of medical text data as positive, negative or uncertain with respect to the first medical term.

The processing circuitry may be configured to receive a classification of the portion of medical text data from an expert user. The processing circuitry may be configured to determine the template and the classification label using the classification received from the expert user.

The template may be verified by an expert user.

The processing circuitry may be configured to receive from the expert user a set of medical terms for which the template is valid. The processing circuitry may be configured to specify the second medical term using the set of medical terms.

The portion of medical text data may further comprise a third medical term having a relationship with the first medical term. The processing circuitry may be further configured to specify a fourth medical term having a relationship with the second medical term, wherein the relationship between the second and fourth medical terms corresponds to the relationship between the first and third medical terms.

The first and third medical terms may be findings. The second and fourth medical terms may be impressions.

The processing circuitry may be further configured to receive a set of known relationships between medical terms. The processing circuitry may be further configured to specify the second and fourth medical terms such that the relationship between the second and fourth medical terms is valid.

The determining of the template may be in response to receiving, by the processing circuitry, a previous classification of the portion of medical image data that was obtained by processing the portion of medical image data using the medical text processing model; and receiving, by the processing circuitry and from an expert user, an indication that the previous classification was incorrect.

The processing circuitry may be further configured to determine at least one counterfactual template associated with a different classification label to the classification label associated with the template. The processing circuitry may be further configured to generate further synthetic text data using the at least one counterfactual template.

The different classification label may be an opposite classification label. The classification label associated with the template may be a first one of positive, negative or uncertain and the different classification label may be a second, different one of positive, negative or uncertain.

The first medical term may comprise an entity. The second medical term may comprise a further entity.

The first medical term may comprise a finding. The second medical term may comprise a further finding.

The first medical term may comprise an impression. The second medical term may comprise a further impression.

The processing circuitry may be further configured to store a plurality of further templates without storing corresponding portions of medical text data from which the further templates were derived. The processing circuitry may be further configured to use the stored plurality of templates to synthesize further text data. The processing circuitry may be further configured to train the medical text processing model on the further synthesized text data.

The processing circuitry may be further configured to use the stored plurality of further templates to add new tasks and distributions to the medical text processing model.

The processing circuitry may be further configured to create a combined template by combining the template with a further template. The processing circuitry may be further configured to synthesize text data using the combined template. The processing circuitry may be further configured to train the medical text processing model using the text data that was synthesized using the combined template.

In a further aspect, which may be provided independently, there is provided a medical information processing method comprising: receiving a portion of medical text data; determining a template corresponding to the portion of medical text data, a classification label associated with the template, and a first medical term included in the portion of medical text data; specifying a second medical term which is different from the first medical term, wherein the second medical term is related to the first medical term; and generating synthetic text data based on the second term by inserting the second medical term into the template.

The medical information processing method may further comprise training the medical text processing model using the synthetic text data and the classification label.

In a further aspect, which may be provided independently, there is provided a system where we synthesize text data via templates which consist of a text sentence and associated labels. The templates take entities as inputs. The entities may have one or more known synonyms that can be input to the template. The synonyms may not have been previously encountered in the real data but may be derived from expert knowledge, including knowledge bases. The synthesized data can be used with or without real text data to train a machine learning model.

Templates may be created to include examples of the rules decided and followed by expert annotators, in order to teach the model about domain-specific or task-specific language interpretation. Templates may be created to include examples of relationships between entities, to teach the model about known relationships between entities. The known relationships may be automatically extracted from a knowledge graph.

Template creation may be one step in an offline or online active learning system, and users may provide feedback on which cases are misclassified, for which an algorithm is trained to derive the templates or additional synonyms required to automatically to solve this type of case.

Templates may be proposed as counterfactual sets. The user may provide further feedback on the accuracy of a template. The user may additionally specify which entities a template is valid for.

Template creation may be used to enable continual learning for a text Al algorithm. A template bank may act as a memory to continuously provide synthesized data that exhibits the key patterns which are important for classification. The template bank may be used to add new tasks and distributions as observed in future populations, even with few or no extra annotation.

The system may be for the classification of radiology reports. The entities may be findings and impressions reported by the radiologist. The synonyms may be extracted from UMLS (Unified Medical Knowledge System) or similar knowledge graph. The relationships may be finding->impression links obtained via expert knowledge.

In a further aspect, which may be provided independently, there is provided a medical information processing apparatus for prosecuting a processing by using a medical text processing model, comprising processing circuitry; receive medical text data, determine a first term (term of interest) included in the medical text data, a template corresponding to the first term, and a label corresponding to the template, specify a second term which is different from the first term, wherein the second term is relating to the first term, generate synthetic text data based on the second term, train the medical text processing model based on the synthetic text data and the label.

The synthetic text data may be generated by replacing the first term included in the template to the second term.

The processing circuitry may be further configured to: determine the first term, the template, and the label in each predetermined data distribution group.

The second term may be a synonym of the first term.

Features in one aspect or embodiment may be combined with features in any other aspect or embodiment in any appropriate combination. For example, apparatus features may be provided as method features and vice versa.

### BRIEF DESCRIPTION OF THE DRAWINGS

Embodiments are now described, by way of non-limiting example, and are illustrated in the following figures, in which:

FIG. 1 is a schematic illustration of a model training process;

FIG. 2 is a schematic illustration of an apparatus in accordance with an embodiment;

FIG. 3 is a flow chart illustrating in overview a text annotation process in accordance with an embodiment;

FIG. 4 shows an example of a radiology report;

FIG. 5 is a flow chart illustrating in overview a data synthesis process in accordance with an embodiment;

FIG. 6 shows an exemplary list of findings and an exemplary list of impressions;

FIG. 7 shows examples of simple templates;

FIG. 8 shows examples of permuted templates;

FIG. 9 shows an example of a combined template;

FIG. 10 shows examples of protocol-derived templates;

FIG. 11 shows examples of incorrect predictions and derived templates;

FIG. 12 is a flow chart illustrating in overview a method of training a model in accordance with an embodiment;

FIG. 13 is a flow chart illustrating in overview a method of creating or amending templates using expert input, in accordance with an embodiment; and

FIG. 14 is a flow chart illustrating in overview a method of automatic template inference in accordance with an embodiment.

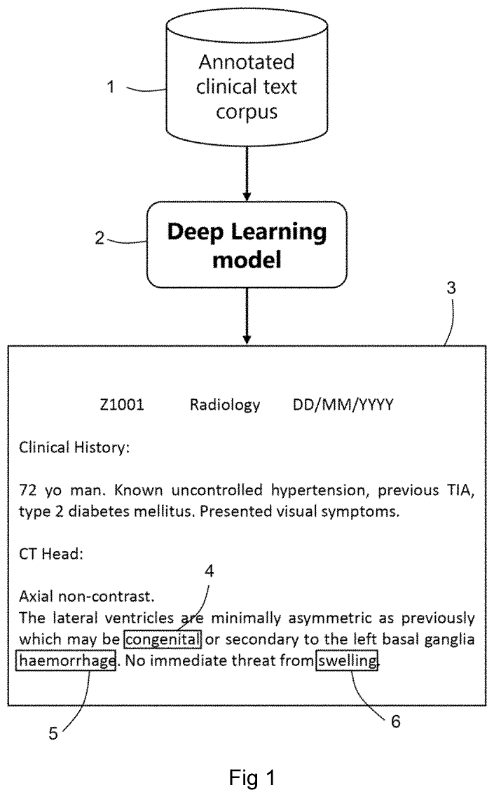

### DETAILED DESCRIPTION

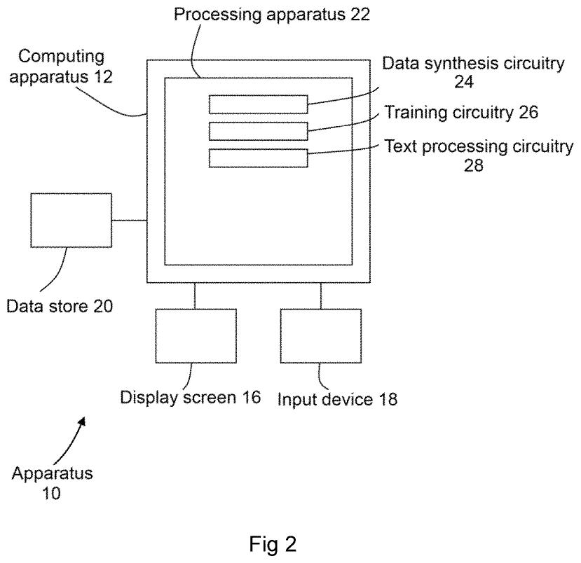

An apparatus 10 according to an embodiment is illustrated schematically in FIG. 2. The apparatus 10 may be referred to as a medical information processing apparatus. The apparatus 10 is configured to train a medical text processing model to process medical text data to predict an output. The medical text data may comprise, for example, clinical notes. In other embodiments, the apparatus 10 may be configured to train a model to process any appropriate text, which may not be medical.

In the embodiment of FIG. 2, the apparatus 10 is also configured to use the trained model to predict the output on previously unseen text data. In other embodiments, a first apparatus may be used to train the model and a second, different apparatus may use the trained model to predict the output. In further embodiments, any apparatus or combinations of apparatuses may be used.

The model may comprise any suitable machine learning model, for example a neural network.

The apparatus 10 comprises a computing apparatus 12, which in this case is a personal computer (PC) or workstation. The computing apparatus 12 is connected to a display screen 16 or other display device, and an input device or devices 18, such as a computer keyboard and mouse.

The computing apparatus 12 receives medical text data from a data store 20. In alternative embodiments, computing apparatus 12 receives medical text data from one or more further data stores (not shown) instead of or in addition to data store 20. For example, the computing apparatus 12 may receive medical text data from one or more remote data stores (not shown) which may form part of an Electronic Medical Records (EMR) or Electronic Health Records (EHR) system or Picture Archiving and Communication System (PACS).

Computing apparatus 12 provides a processing resource for automatically or semi-automatically processing medical text data. Computing apparatus 12 comprises a processing apparatus 22. The processing apparatus 22 comprises data synthesis circuitry 24 which is configured to use templates to perform data synthesis; training circuitry 26 which is configured to train a machine learning model using the synthesized data; and text processing circuitry 28 which is configured to apply the trained model to unseen text data.

In the present embodiment, the circuitries 24, 26, 28 are each implemented in computing apparatus 12 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 2 for clarity.

The apparatus 10 of FIG. 2 is configured to perform methods as described in the following.

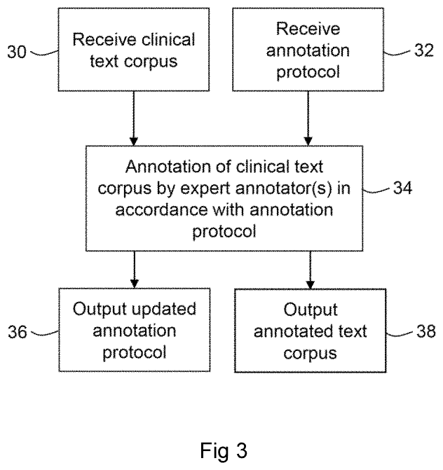

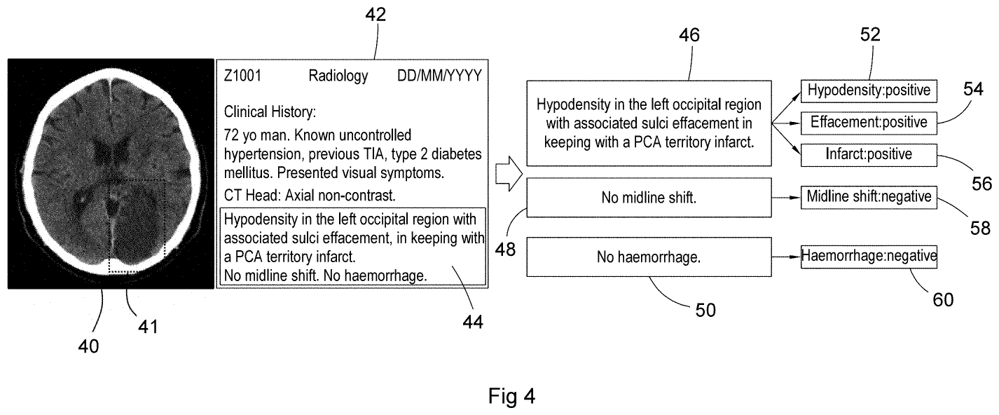

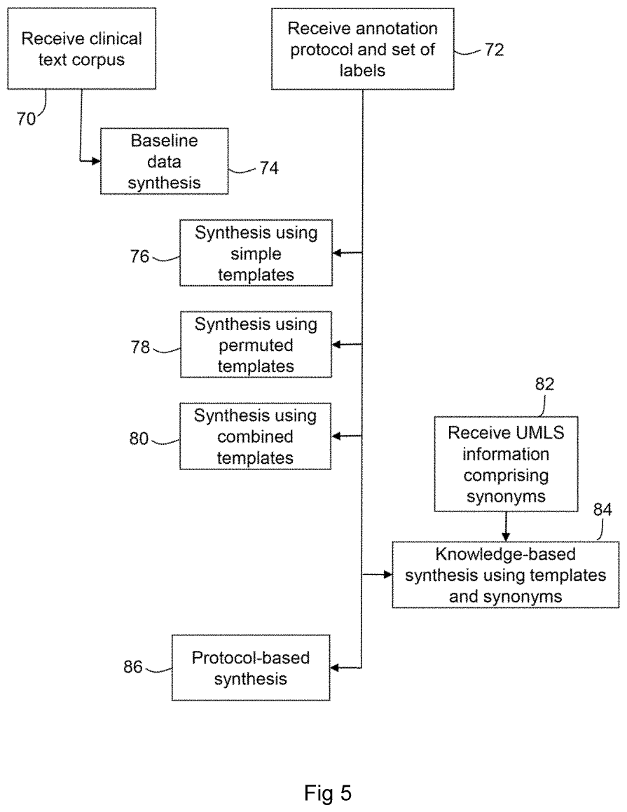

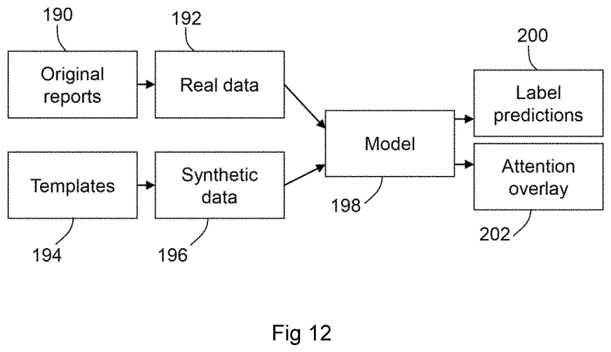

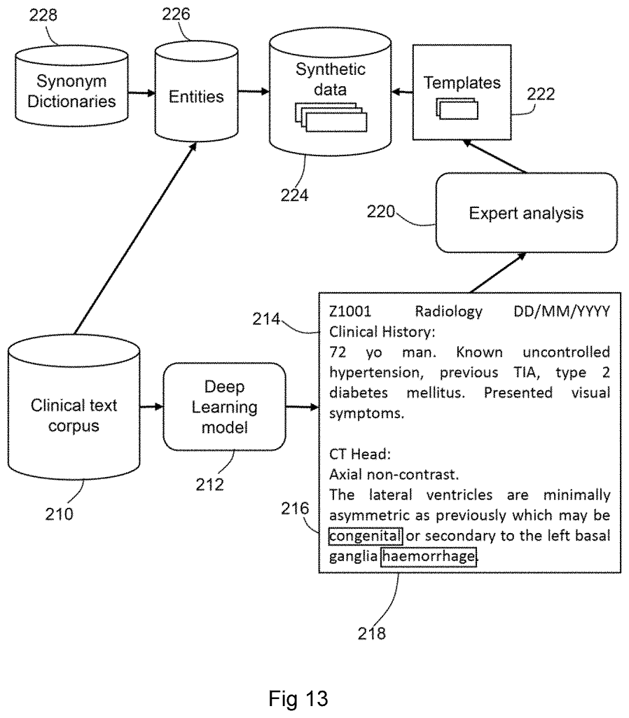

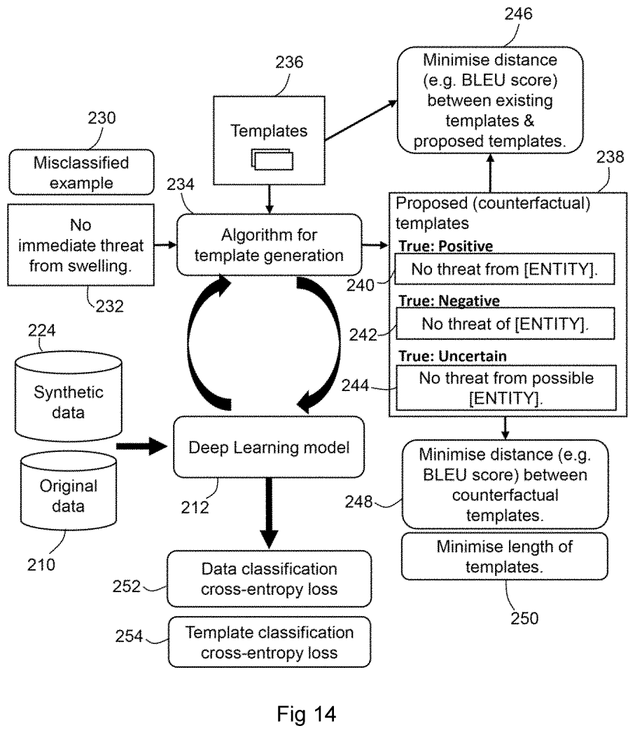

In the present embodiment, apparatus 10 is configured to perform a text annotation process as described below with reference to FIGS. 3 and 4; to perform a text synthesis process as described below with reference to FIGS. 5 to 11, which includes generating a plurality of templates; to perform a model training process as described below with reference to FIG. 12; to perform an active learning process as described below with reference to FIG. 13; and to perform automatic template inference as described below with reference to FIG. 14.

In other embodiments, different apparatus may be used to perform different processes, or parts of processes, of those described below. For example, a first apparatus may be used to perform text annotation, a second apparatus may be used to perform template generation, a third apparatus may be used to perform text data synthesis, a fourth apparatus may be used to train a deep learning model and a fifth learning model may apply the deep learning model to new text. Any suitable combination of apparatuses may be used.

FIG. 3 is a flow chart illustrating in overview a text annotation process that is performed by the data synthesis circuitry 24 of apparatus 10 in accordance with an embodiment. In other embodiments, different circuitry or a different apparatus may be used to perform the text annotation, and the resulting annotated text may subsequently be received by the data synthesis circuitry 24.

At stage 30, a clinical text corpus is received by the data synthesis circuitry 24 from the data store 20 or from any suitable data store. The clinical text corpus comprises a plurality of text documents, for example a plurality of radiology reports.

At stage 32, the data synthesis circuitry 24 receives an annotation protocol. The annotation protocol comprises a set of rules for expert annotators to use in annotating documents of the clinical text corpus.

At stage 34, the clinical text corpus is annotated by one or more expert annotators in accordance with the annotation protocol. The expert annotators may annotate the clinical text corpus by providing any suitable input via any suitable input device 18, for example by typing using a keyboard.

The expert annotators may be any persons with suitable medical knowledge, for example radiologists, clinicians or other trained annotators. In other embodiments, any one or more human annotators may annotate the clinical text corpus.

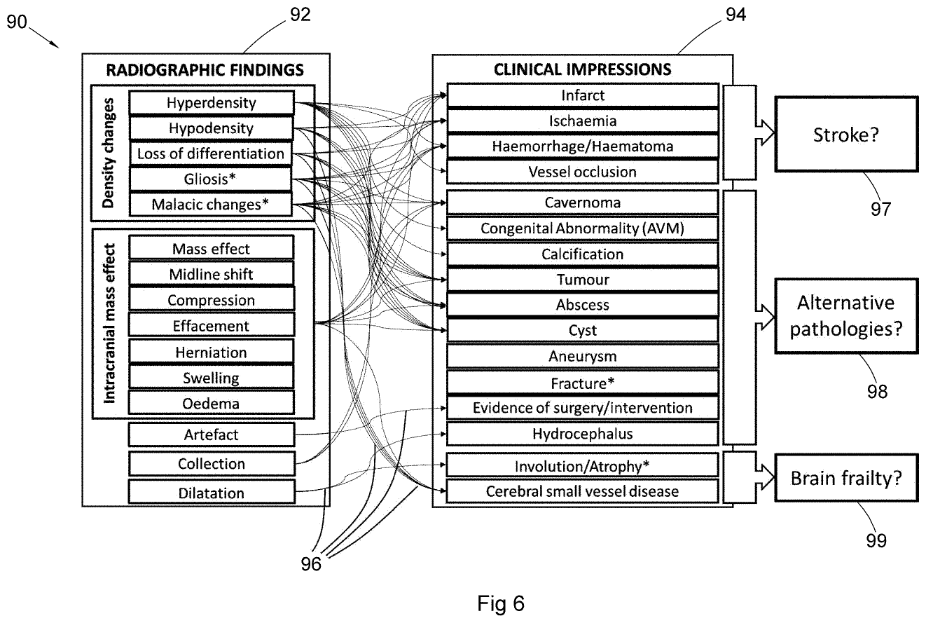

The expert annotators perform a classification of a set of labels. In the present embodiment, the labels are medical labels. Each label comprises a medical term. Some of the medical terms are findings and some are impressions. Some of the medical terms may be both findings and impressions. Contents of an exemplary set of medical labels are described further below with reference to FIG. 6.

The expert annotators consider each sentence of each text document of the clinical text corpus. The expert annotators may select for annotation any sentence that contains medically relevant information. Sentences that do not contain any medical relevant information may be omitted. In other embodiments, only a subset of sentences in the clinical text corpus may be selected for annotation and some medically relevant sentences may not be annotated.

In further embodiments, any suitable portions of text may be considered, which may or may not be sentences. For example, the portions of text may be parts of sentences, or pairs or groups of sentences. The portions of text may be text fragments.

For each sentence that is to be annotated, the expert annotators determine which medical labels are mentioned in the sentence. Where a medical label is mentioned in the sentence, the expert annotators classify the medical label as positive (finding or impression is present), negative (finding or impression is not present) or uncertain (presence of finding or impression is uncertain). Positive, negative and uncertain may be referred to as classification labels or certainty classes. For each sentence, the annotators may assign a respective classification label to each medical label that is mentioned in the sentence. Classification labels for medical labels that are not mentioned in the sentence may be left blank.

The expert annotators may input the classification labels using any suitable input device 18 or input method.

In some circumstances, the classification may be straightforward. For example, the sentence may directly say that ‘hyperdensity is present’ or ‘there is hyperdensity’. It may then be straightforward for an annotator to assign a classification level of positive to the medical label of hyperdensity.

However, as mentioned above, in some sentences there may be ambiguous wording that requires expert consensus to interpret. Different experts may not agree on how a given sentence to be classified. Rules are therefore defined in the annotation protocol to define a consistent classification of particular types of sentence structure or content.

In the embodiment of FIG. 3, rules of the annotation protocol are changed or augmented during the process of annotation. Existing rules may be changed or new rules may be added. For example, new rules may be added if new findings or impressions or new sentence structures are found by the annotators.

The expert annotators may input changes to the annotation protocol using any suitable input device 18 and input method. The data synthesis circuitry 24 updates the stored annotation protocol based on the input of the expert annotators.

At stage 36, the data synthesis circuitry 24 outputs an updated annotation protocol. The updated annotation protocol includes any changes to the annotation protocol that the experts have made during the annotation process of stage 34. The updated annotation protocol may be stored in data store 20 or in any suitable data store.

In other embodiments, there may be no change in the annotation protocol during the annotation process of FIG. 34. In such embodiments, no updates are made to the annotation protocol and stage 36 is omitted.

At stage 38, the data synthesis circuitry 24 outputs an annotated version of the clinical text corpus. The annotated version of the clinical comprises, for each annotated sentence, a respective classification label for each medical label that is mentioned in the sentence. The annotated clinical text corpus may be stored in data store 20 or in any suitable data store.

FIG. 4 shows an example of a radiology report 42 that is similar to a radiology report which may be contained in a clinical text corpus. The particular example shown in FIG. 4 has been synthesized but has a similar format to real radiology data. The radiology report 42 is annotated by expert annotators as described above with reference to FIG. 3.

The radiology report 42 is associated with at least one medical image. In the example of FIG. 4, the at least one medical image comprises a slice 40 from a CT scan. In the slice 40 from the CT scan, there is a visible darker patch indicating an infarct. The visible darker patch is highlighting in the medical image of slice 40 by box 41.

In an annotation process, a region 44 of relevant sentences is identified in the radiology report 42. In the example shown, relevant sentences 46, 48, 50 are manually filtered from the radiology report. The extracted sentences 46, 48, 50 are each annotated by the expert annotators.

A set of boxes 52, 54, 56, 58, 60 indicate medical labels that are annotated in the sentences 46, 48, 50, and the classification labels that are annotated for each of the medical labels.

Sentence 46 reads ‘Hypodensity in the left occipital region with associated sulci effacement in keeping with a PCA territory infarct’. Boxes 52, 54, 56 relate to the medical terms hypodensity, effacement and infarct respectively in sentence 46. The association of boxes 52, 54, 56 with sentence 46 show that it has been determined by an expert annotator that sentence 46 mentions hypodensity, effacement and infarct. Hypodensity in box 52 is given a classification label of positive. Effacement in box 54 is given a classification label of positive. Infarct in box 56 is given a classification label of positive.

Sentence 48 reads ‘No midline shift’. Box 58 relates to the medical term midline shift in sentence 48. The association of box 58 with sentence 48 shows that the expert annotator has identified midline shift in sentence 48. Midline shift in box 58 is given a classification label of negative.

Sentence 50 reads ‘No haemorrhage’. Box 60 is used to highlight the medical term haemorrhage. The association of box 60 with sentence 50 shows that the expert annotator has identified haemorrhage in sentence 50. Haemorrhage in box 60 is given a classification label of negative.

FIG. 5 is a flow chart illustrating in overview a method of generating synthetic data in accordance with an embodiment. The method is performed by the data synthesis circuitry 24 of apparatus 10. In other embodiments, the method of FIG. 5 may be performed by any suitable circuitry or apparatus.

At stage 70 of FIG. 5, the data synthesis circuitry 24 receives a set of training data. The set of training data comprises or is derived from an annotated clinical text corpus, for example the annotated clinical text corpus that was output at stage 38 of FIG. 3. The set of training data comprises a plurality of portions of medical text data, which in the embodiment of FIG. 5 is a plurality of sentences. Each sentence has been annotated as described above, such that each medical term in a sentence has an associated classification label. In the embodiment of FIG. 4, each medical term is a finding or impression. Each sentence is associated with a patient, where one or more sentences may be associated with each patient. The associated classification label classifies the finding or impression as positive if the sentence indicates that the finding or impression is present in the patient, negative if the sentence indicates that the finding or impression is present in the patient, or uncertain if the sentence indicates that it is uncertain whether the finding or impression is present in the patient.

At stage 72, the data synthesis circuitry 24 receives an annotation protocol, which may correspond to the annotation protocol of stage 32 of FIG. 3, or to the updated annotation protocol of stage 38 of FIG. 3. The data synthesis circuitry 24 also receives a set of medical labels, which in the embodiment of FIG. 5 comprise a plurality of findings 92 and a plurality of impressions 94. The set of medical labels may form part of the annotation protocol.

In the present embodiment, the data synthesis circuitry 24 receives a knowledge graph 90 relating a set of findings 92 to a set of impressions 94. The knowledge graph 90 may form part of or be associated with the annotation protocol.

FIG. 6 shows an example of a knowledge graph 90. A labelling system has been turned into a knowledge graph showing the links between findings and impressions.

The knowledge graph 90 comprises a set of radiographic findings 92 and a set of clinical impressions 94, which in the present embodiment are the findings 92 and impressions of the set of medical labels. Labels that are marked with an asterisk * are labels that fit both the finding and impression category. Links between findings 92 and impressions 94 are shown by a set of lines 96.

The knowledge graph 90 further shows a plurality of possible conclusions based on impressions 94. The possible conclusions comprise stroke 97, alternative pathologies 98 and brain frailty 99.

At stage 74, the data synthesis circuitry 24 performs a baseline data synthesis comprising random deletion and random insertion. The baseline data synthesis is performed using sentences from the training dataset. In the random deletion approach, a synthetic sentence is created for each original sentence in the training dataset by deleting a single randomly selected word each time. The random insertion approach similarly creates one synthetic sentence for each original sentence in the training dataset. In the random insertion approach, a randomly selected stop word is inserted. Stop words are the most frequent words used in a language such as “a”, “for”, “in” or “the”.

At stage 76, the data synthesis circuitry 24 performs a data synthesis using a plurality of simple templates. Templates are text structures that use pre-written texts with placeholders for certain words. In the text synthesis of stage 76, each template is a sentence in which a medical term has been replaced by a placeholder word. For example, one template is ‘There is [ENTITY]’. ‘There is’ may be considered to be text obtained from of an original sentence, for example ‘There is hyperdensity’. ENTITY is a placeholder term that takes the place of any suitable medical term, for example any suitable finding or impression.

In other embodiments, a template may represent any portion of text data, for example a sentence fragment or a pair or group of sentences. Any word in the portion of text data may be replaced by a corresponding placeholder term. Any number of words may be replaced by placeholders. Any suitable format for creating and storing templates may be used.

Each template has an associated classification label. The classification label is positive, negative or uncertain depending on the sentence on which the template is based. For example, the template ‘There is [ENTITY]’ is associated with a classification label that is positive.

A goal of the data synthesis of stage 76 is to provide an example for every entity class, where the classes include each certainty value for each medical label.

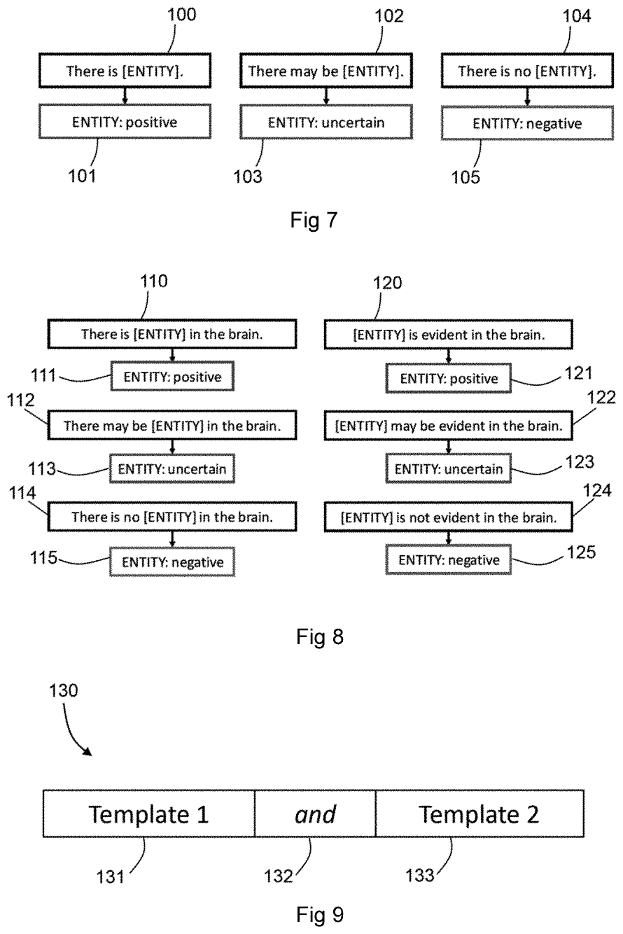

FIG. 7 shows the simple templates of stage 76 of the present embodiment. Square brackets are used to denote a placeholder text. The term ENTITY is a placeholder text which may be replaced with any suitable medical term, for example any suitable finding or impression in the set of medical labels received at stage 72.

Template 100 comprises the text ‘There is [ENTITY]’. Template 100 is associated with a classification 101 of ENTITY as positive.

Template 102 comprises the text ‘There may be [ENTITY]’. Template 102 is associated with a classification 103 of ENTITY as uncertain.

Template 104 comprises the text ‘There is no [ENTITY]’. Template 104 is associated with a classification 105 of ENTITY as negative.

Each of the findings 92 and impressions 94 of FIG. 6 is inserted into the placeholder position of each of the templates 100, 102, 104 to replace the placeholder [ENTITY].

The data synthesis circuitry 24 adds one synthetic sentence for each of the uncertainty classes and labels which results in at least one example for each label and certainty class combination. The use of templates to generate synthetic sentences may enable learning of combinations that are not present in the original training data. The use of templates to generate synthetic sentences may allow for zero-shot or few-shot learning.

An output of stage 76 is a set of synthetic sentences that have been generated using the simple templates of FIG. 6. The synthetic sentences comprise every combination of the templates 100, 102, 104 with each of the findings 92 and impressions 94 listed in FIG. 6.

At stage 78, the data synthesis circuitry 24 performs a data synthesis using a plurality of permuted templates. The data synthesis of stage 78 adds further synthetic sentences in which the position of the medical label (for example, the finding 92 or impression 94) within the sentence is changed. A goal of stage 78 is to provide examples with entities at different positions in the sentence.

FIG. 8 shows some permuted templates of the present embodiment. Square brackets are used to indicate a placeholder, and [ENTITY] is used as a placeholder for a medical term. The number of the permuted templates that are classified as positive is the same as the number that are classified as negative, and the same as the number that are classified as uncertain. A balanced set of synthetic data may be created by using equal numbers of positive, negative and uncertain templates.

Template 110 comprises the text ‘There is [ENTITY] in the brain’. Template 110 is associated with a classification 111 of ENTITY as positive.

Template 112 comprises the text ‘There may be [ENTITY] in the brain’. Template 112 is associated with a classification 113 of ENTITY as uncertain.

Template 114 comprises the text ‘There is no [ENTITY] in the brain’. Template 114 is associated with a classification 115 of ENTITY as negative.

Template 120 comprises the text ‘[ENTITY] is evident in the brain’. Template 120 is associated with a classification 121 of ENTITY as positive.

Template 122 comprises the text ‘[ENTITY] may be evident in the brain’. Template 122 is associated with a classification 123 of ENTITY as uncertain.

Template 124 comprises the text ‘[ENTITY] is not evident in the brain’. Template 124 is associated with a classification 125 of ENTITY as negative.

In other embodiments, any suitable permuted templates may be used. The templates may be based on any suitable portion of text. The placeholder for the medical terms may be positioned at any suitable point within each sentence.

An output of stage 78 is a set of synthetic sentences that have been generated using the permuted templates.

By using simple and/or permuted templates to create synthetic sentences using all of the labels in the set of labels, training data may be created that includes examples of all of the labels. The training data may include positive, negative and uncertain examples even for labels that are rare in the real training data corpus.

The templates of stages 76 and 78 each include the placeholder text [ENTITY] which can be replaced with any medical term, for example any finding or impression. The templates take entities as inputs. In other embodiments, the inputs that replace placeholder text may be more limited, for example by specifying [FINDING] or [IMPRESSION] or by limiting inputs to a predetermined set of medical terms.

At stage 80, the data synthesis circuitry 24 uses a combination template 130 as shown in FIG. 9 to generate more complex sentences using a selection of all previous synthesized sentences. The previous synthesized sentences generated at stages 76 and 78 may be referred to as basic synthetic sentences. A more complex sentence generated at stage and related may be referred to as a combined sentence.

A first part 131 of the combined sentence is a basic synthetic sentence generated using any of the templates of stage 76 or stage 78, and is referred to in FIG. 9 as Template 1. A second part 132 of the combined sentence is the word ‘and’. A third part 133 of the combined sentence is another basic synthetic sentence generated using any of the templates of stage 76 at stage 78, and is referred to in FIG. 9 as Template 2.

Any number of combined sentences may be created up to a maximum of the square of the number of basic synthetic sentences. In the present embodiments, 200 combinations are randomly sampled.

If a random selection of basic synthetic sentences results in Template 1 and Template 2 having the same label but with different certainty classes, the following precedence rule defined in the annotation protocol is used to label the sentence with a single label:

positive>negative>uncertain>blank

The combined templates of stage 80 aim to provide examples of entities with different modifiers present in the same sentence.

An example sentence generated by the combined template 130 is “There is hyperdensity in the brain and there is no infarct” which would be labelled as positive hyperdensity and negative infarct.

An issue has been observed in some existing models that when words such as “not” are detected, the sentence may be classified as negative without taking context into account. The model may learn from syntactic heuristics. For example, “This exerts only minimal local mass effect and no distant brain herniation.” should be labelled as positive mass effect and negative herniation but the model may learn to label both entities as negative.

To address this, the combined template synthesis of stage 80 deliberately creates many counter-examples by combining templates very simply with the word “and” in between.

In other embodiments, any suitable combination template may be used to combine two or more basic synthetic sentences. In some embodiments, a first synthetic sentence is obtained using Template 1 and a second synthetic sentence is obtained using Template 2, and the synthetic sentences are combined to obtain a combined sentence. In other embodiments, Template 1 and Template 2 are combined to give a single combined template. Appropriate medical terms are then inserted into the placeholder positions of the combined template.

In further embodiments, one or more further combination templates may be used. A further combination template may use a different combining term in place of ‘and’. For example, the combining term may be a comma, the word ‘while’ followed by a comma, the word ‘plus’, the word ‘also’, the word ‘furthermore’, the word ‘alongside’, the words ‘in addition’ or the words ‘along with’.

At stage 82, the data synthesis circuitry 24 obtains a set of synonyms from one or more existing knowledge bases. The set of synonyms may also be referred to as a synonym list or synonym dictionary.

The set of synonyms comprises one or more synonyms for each medical label of the set of medical labels received at stage 72. Each entity may have one or more known synonyms. The synonyms may not have been encountered in the real data that is used to train a model, but are derived from expert knowledge.

In the present embodiment, the existing knowledge base from which expert knowledge is obtained is the Unified Medical Language System (UMLS). In other embodiments, the set of synonyms may be obtained from any suitable knowledge source, for example any suitable database, knowledge base, knowledge graph or ontology. The set of synonyms may be stored in the data store 20 or in any suitable data store. The set of synonyms may be received along with the knowledge graph 90 at stage 72.

At stage 84, the data synthesis circuitry 24 uses the templates of stages 76, 78 and/or 80 to synthesize further synthetic sentences in which synonyms for the medical labels 92, 94 are inserted into the placeholder [ENTITY]. For example, the template ‘There is [ENTITY]’ was populated at stage 74 with all of the findings 92 and impressions 94 in the knowledge graph 90. At stage 84, the template ‘There is [ENTITY]’ is populated with each of the synonyms for the findings 92 and impressions 94 that is listed in the set of synonyms.

Templates are used to inject expert knowledge into the training data. At stage 84, the expert knowledge is knowledge that is obtained from the knowledge base. Synonyms of labels may be mined from existing knowledge bases and inserted into templates.

Some of the labels, such as tumour and infection, have many different subtypes which may not be exhaustively mentioned in the training data received at stage 70. In one exemplary data set of stroke patients, tumours are one of the rarest labels in the data set and infection is not present in the training data at all. By injecting the synonyms for the findings 92 and impressions 94, the model can be trained to pick up on different mentions of tumours in a simple and automated way. The model may be trained to pick up on many different findings or impressions using synonyms that are not present in the training data.

The use of synonyms in creating synthetic data may be particularly helpful for broad classes such as “evidence of intervention” (examples of synonyms are “craniotomy”, “tube”, “shunt”) and “infection” (examples of synonyms are “osteomyelitis”, “cerebritis”, “ventriculitis”).

An output of stage 84 is a set of synthetic sentences created using one or more synonym dictionaries. In the present embodiment, the synonym dictionary is derived from the UMLS. In other embodiments, any suitable synonym dictionary may be used. An existing knowledge base is used to insert unseen synonyms into the templates.

At stage 86, the data synthesis circuitry 24 derives further templates from the annotation protocol received at stage 72. These further templates may be described as protocol-derived templates which aim to provide examples of protocol-specific rules. The protocol-derived templates make use of expert knowledge which was used to develop the annotation protocol. The data synthesis circuitry 24 creates further synthetic sentences by populating the protocol-derived templates.

In the present embodiment, rules were developed in the annotation protocol for how to interpret ambiguous language. The ambiguous language was language that the annotators found difficult to label. In the annotation process described in FIG. 3, new rules are developed during the annotation process as new terms or new sentence structures are encountered.

It has been found that creating a human annotation protocol is difficult. The protocol may be constantly evolved with new data that is labelled. It may therefore be useful to include certain phrases or rules that are included in the protocol in a template, so that the template can be learned by the model. For example, templates may be used for certainty class modifiers such as “suggestive” compared to “suspicious”.

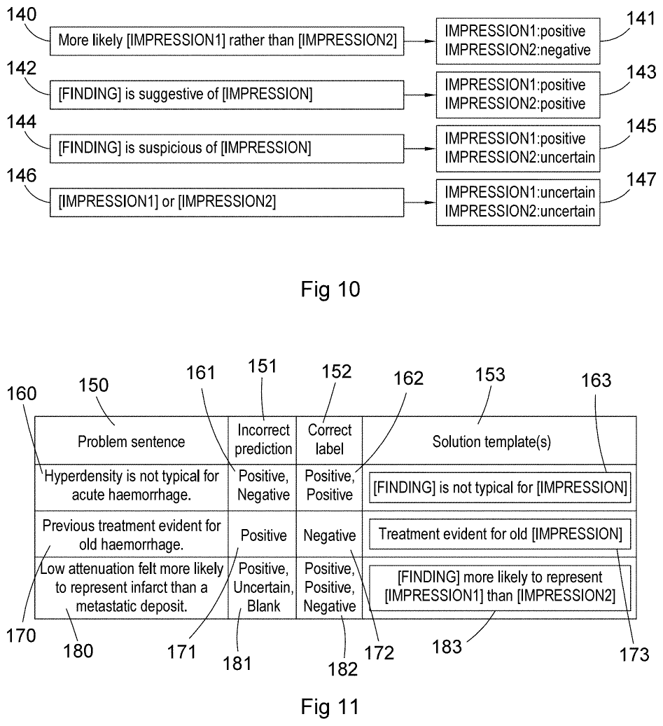

Templates shown in FIG. 10 have been derived from an annotation protocol. For example, the templates may be extracted from a list of templates that are stored as part of the annotation protocol. The templates may be generated by processing a set of annotation instructions. The templates include examples of rules decided and followed by expert annotators. By including examples of text synthesized using the rules of the annotation protocol, a model may be taught about domain-specific or task-specific language interpretation.

Template 140 comprises the text ‘More likely [IMPRESSION1] rather than [IMPRESSION2]’. The correct classification 141 for the template 140 according to the annotation protocol is that [IMPRESSION1] is classified as positive, and [IMPRESSION2] is classified as negative. The data circuitry 24 generates synthetic sentences by replacing [IMPRESSION1] with a first impression from the knowledge graph 90 or list of medical labels, and replacing [IMPRESSION2] with a second impression from the knowledge graph 90 or list of medical labels.

In some embodiments, any first impression and second impression may be used to populate template 140 by inserting the first impression at the first placeholder position to replace [IMPRESSION1] and inserting the second impression at the second placeholder position to replace [IMPRESSION2]. In other embodiments, only selected pairs of first and second impressions may be used to populate template 140. For example, only impressions that may be easily confused may be included. Only impressions that are considered similar may be included. Relationships between impressions or a list of suitable pairs of impressions may be obtained from any suitable knowledge source.

Template 142 comprises the text ‘[FINDING] is suggestive of [IMPRESSION]’. The correct classification 143 for the template 142 according to the annotation protocol is that [FINDING] is classified as positive and [IMPRESSION] is classified as positive.

Template 144 comprises the text ‘[FINDING] is suspicious of [IMPRESSION]’. The correct classification 145 for the template 144 according to the annotation protocol is that [FINDING] is classified as positive and [IMPRESSION] is classified as uncertain.

The data synthesis circuitry 24 generates synthetic sentences from template 142 and template 144 by inserting a finding at the position of the first placeholder [FINDING] and inserting an impression at the position of the second placeholder [IMPRESSION]. To populate templates 142 and 144, the data synthesis circuitry 24 may make use of relationships between findings 92 and impressions 94 such as those included in knowledge graph 90 and shown by lines 96 in FIG. 6. By using known relationships between findings 92 and impressions 94, the data synthesis circuitry 24 may avoid creating synthetic sentences that are unlikely to be found in real radiology reports, for example synthetic sentences that describe an unrealistic inference of an impression based on a finding.

Template 146 comprises the text ‘[IMPRESSION1] or [IMPRESSION2]’. The correct classification 147 for the template 146 according to the annotation protocol is that [IMPRESSION1] is classified as uncertain and [IMPRESSION2] is classified as uncertain. The data circuitry 24 generates synthetic sentences by replacing [IMPRESSION1] with a first impression and replacing [IMPRESSION2] with a second impression. Relationships between impressions or a list of suitable pairs of impressions may be obtained from any suitable knowledge source.

Data is synthesized to include examples between entities, to teach a model about known relationships between entities. The known relationships may be automatically extracted from a knowledge graph or may be obtained using any suitable method.

FIG. 11 shows some examples of sentences that are considered to be difficult to classify, and how templates can be created to teach the model the same rules. The templates of FIG. 11 are created from example sentences. In other embodiments, templates may be created from any suitable portions of text data.

Each example of FIG. 11 comprises a problem sentence 150 which is considered to be difficult to classify. The problem sentence 150 may be identified by an expert, or in any suitable manner. The problem sentence 150 is a sentence for which an incorrect prediction 151 has been obtained. Correct labels 152 have then been obtained in accordance with the annotation protocol. A template 153 is derived from the problem sentence 150. In some embodiments, the template 153 is created by an expert. In other embodiments, the template 153 is created automatically by the data synthesis circuitry 24. The automatically-created template 153 may then be verified by an expert.

The incorrect prediction 151, correct labels 152 and template 153 are shown in FIG. 11. The incorrect prediction 151 may be a prediction that is obtained from a trained model that has not yet been trained on the template 153.

A first example sentence 160 is ‘Hyperdensity is not typical for acute haemorrhage’. An incorrect prediction 161 for the first example sentence 160 is to classify hyperdensity as positive and haemorrhage as negative. For example, a model may classify haemorrhage as negative because of the presence of the word ‘not’. Correct labels 162 classify hyperdensity as positive and haemorrhage as positive.

The data synthesis circuitry 24 generates a template 163 based on the first example sentence 160. To generate the template 163, the data synthesis circuitry 24 identifies two medical terms within the example sentence. The medical terms may be identified using the list of medical terms received at stage 72, or manually identified by an expert annotator. In other embodiments, the medical terms may also be identified using the list of synonyms received at stage 82, or using any appropriate knowledge source. In example sentence 160, one medical term is ‘Hyperdensity’ and another medical term is ‘Haemorrhage’.

The data synthesis circuitry 24 replaces each medical term with a corresponding placeholder term. In the case of the first example sentence 160, the data synthesis circuitry 24 replaces the medical term ‘Hyperdensity’ with the placeholder text FINDING and replaces the medical term ‘Haemorrhage’ with the placeholder text IMPRESSION.

The template 163 generated based on the first example sentence 160 is ‘[FINDING] is not typical for [IMPRESSION]’.

The data synthesis circuitry 24 generates synthetic sentences by replacing the placeholder text [FINDING] with a finding from the list of medical terms and replacing the placeholder text [IMPRESSION] with an impression from the list of medical terms.

For example, the data synthesis circuitry may replace [FINDING] with ‘loss of differentiation’ and [IMPRESSION] with ‘tumour’. ‘Loss of differentiation’ is related to ‘Hyperdensity’ in the original sentence 160 in that loss of differentiation and hyperdensity are both findings. ‘Hyperdensity’ is first replaced with the placeholder text [FINDING] and then with the medical term ‘loss of differentiation’. ‘Tumour’ is related to ‘haemorrhage’ in the original sentence 160 in that tumour and haemorrhage are both impressions.

‘Haemorrhage’ is first replaced with the placeholder text [IMPRESSION] and then with the medical term ‘ tumour’.

The data synthesis circuitry 24 may use the knowledge graph 90 or any suitable source of knowledge to identify suitable pairings of a finding and an impression. Information about pairs of findings and impressions may be provided by an expert annotator. The findings and impressions that are used to populate the template 163 may be chosen such that the synthetic sentences that are generated from template 163 reflect realistic relationships between finding and impression.

A second example sentence 170 is ‘Previous treatment evident for old haemorrhage’. An incorrect prediction 171 for the second example sentence 170 includes a classification of haemorrhage as positive. A correct label 172 according to the annotation protocol is to classify haemorrhage as negative, because the haemorrhage is not current.

The data synthesis circuitry 24 generates a template 173 based on the second example sentence 170. To generate the template 173, the data synthesis circuitry 24 identifies the medical term ‘haemorrhage’ within the example sentence 170. The data synthesis circuitry 24 replaces the medical term ‘haemorrhage’ with the placeholder term ‘IMPRESSION’.

The template 173 generated from the second example sentence 170 is ‘Treatment evident for old [IMPRESSION]’. The data synthesis circuitry 24 generates synthetic sentences by replacing [IMPRESSION] with any impression from the knowledge graph 90. For example, [IMPRESSION] may be replaced by ‘Vessel occlusion’. The term vessel occlusion is related to the original term because both are medical terms that refer to impressions.

In other embodiments, [IMPRESSION] may be replaced with any suitable impression from any suitable data source.

A third example sentence 180 is ‘Low attenuation felt more likely to represent infarct than a metastatic deposit’. An incorrect prediction 181 for the third example sentence 180 is positive for low attenuation, uncertain for infarct, and blank (not mentioned) for metastatic deposit. A correct labelling 182 according to the annotation protocol is to classify low attenuation as positive, infarct as positive, and metastatic deposit as negative.

The data synthesis circuitry 24 generates a template 183 based on the third example sentence 180. To generate the template 183, the data synthesis circuitry 24 identifies three medical terms within the example sentence 180. The three medical terms are ‘low attenuation’, ‘infarct’ and ‘metastatic deposit’. The data synthesis circuitry 24 replaces ‘low attenuation’ with the placeholder term FINDING ; replaces ‘infarct’ with the placeholder term ‘IMPRESSION1’; and replaces ‘metastatic deposit’ with the placeholder term ‘IMPRESSION2’.

The template 183 generated from the third example sentence 180 is ‘[FINDING] more likely to represent [IMPRESSION1] than [IMPRESSION2]’. The data synthesis circuitry 24 generates synthetic sentences by replacing [FINDING], [IMPRESSION1] and [IMPRESSION2] using information from the knowledge graph 90. In other embodiments, information may be obtained from any suitable knowledge source.

The findings and impressions that are used to populate the template 183 may be chosen such that the synthetic sentences that are generated from template 183 reflect realistic relationships between finding and impressions.

In summary, the data synthesis circuitry 24 generates synthetic sentences by inserting appropriate medical terms into placeholder positions in the protocol-derived templates, for example in templates 140, 142, 144, 146, 163, 173 and 183.

Some templates are only valid for certain entities. Expert medical knowledge is used to determine templates that are only valid for certain entities, to produce a valid data distribution for each template. Predetermined data distribution groups may be used. For example, templates may be populated only with entities relating to a certain pathology or to a certain population.

Each template is only populated with entities for which it is valid. For example, it may be necessary for findings and impressions to be paired correctly such that the finding in a sentence could lead to the impression in that sentence.

In other embodiments, knowledge for creating and/or populating the templates may be found in any suitable knowledge source. The knowledge may be obtained from any suitable database, knowledge base, knowledge graph or ontology. The knowledge may be obtained from an expert, for example from one or more of the expert annotators.

Expert knowledge may also be applied in the populating of templates in any of stages 76, 78, 80 and 82.

In the method of FIG. 5, some of the templates are created for use in training a model on rare classes. Some of the templates are created for use in training a model on difficult sentence structures which may be misclassified.

An output of the data synthesis process of FIG. 5 is a set of synthetic data comprising a plurality of synthetic sentences. The plurality of synthetic sentences comprises synthetic sentences generated in stages 74, 76, 78, 80, 84 and 86. In other embodiments, any one or more of stages 74, 76, 78, 80, 84 and 86 may be omitted. Additional data synthesis stages may be added. Data synthesis stages may be combined with other data synthesis stages.

FIG. 12 is a flow chart illustrating in overview a model training method of an embodiment.

At stage 190, the data synthesis circuitry 24 receives a set of original reports, for example a clinical text corpus 30 comprising a plurality of radiology reports 42 as described above with reference to FIGS. 3 and 4. At stage 192, the data synthesis circuitry 24 processes the original reports to obtain real data, for example by extracting one or more sentences 46, 48, 50 from each report. The real data is manually annotated by one or more annotators as described above with reference to FIGS. 3 and 4. The annotation is in accordance with an annotation protocol. The annotation protocol may be updated with new or amended rules during the annotation process.

A result of the annotation process is to associate classification labels with the sentences that are annotated. The classification labels provide a ground truth classification of the sentences.

At stage 194, the data synthesis circuitry 24 receives a set of templates. At least some templates of the set of templates may be derived from the annotation protocol as described above with reference to stage 86 of FIG. 5, and with reference to FIGS. 10 and 11.

At stage 196, the data synthesis circuitry 24 uses the set of templates to generate a set of synthetic data comprising a plurality of synthesized sentences. Each synthesized sentence has an associated classification in accordance with a classification of the template. The classification labels provide ground truth data for classification of the resulting sentences. The data synthesis process of stage 196 may comprise some or all of the data synthesis stages 74, 76, 78, 80, 84 and 86 described above in relation to FIG. 5.

At stage 198, the training circuitry 26 trains a model using both the real data of stage 192 and the synthetic data of stage 196. In the present embodiments, the model is a neural network model, for example a convolutional neural network. In other embodiments, any suitable machine learning model may be used.

The real data comprises a set of sentences and associated classification labels, where the classification labels were obtained by annotation. The synthetic data comprises a set of synthetic sentences and associated classification labels, where the classification labels are in accordance with the templates from which the synthetic sentences were derived.

The model is trained to output a set of label predictions 200. The set of label predictions may comprise predictions of whether each of the set of medical labels on which the model was trained is positive, negative, uncertain, or not mentioned in a given sentence.

In training, sentences from both the real data and the synthetic data are input to the model. Outputs of the model are compared to ground truth data. Errors in the output of the model are fed back to the model.

The model may be trained using any suitable model training method, for example using stochastic gradient descent, Adam (Kingma, D. P.; Ba, J. Adam: A Method for Stochastic Optimization. 3rd International Conference on Learning Representations, ICLR, San Diego, Calif., USA, May 7-9, 2015, Conference Track Proceedings; Bengio, Y.; LeCun, Y., Eds., 2015.) or AdamW (Decoupled Weight Decay Regularization, I Loshchilov, F Hutter, International Conference on Learning Representations (ICLR 2019)).

In the present embodiment, the model is also trained to output an attention overlay 202. In the process of classifying each sentence, the machine learning model determines a respective attention contribution for each of the words in the sentence. The attention contribution for a given word indicates how important that word was to the classifying of the sentence. The attention overlay may comprise an attention vector having as many elements as the number of words in the sentence. The attention vector may comprise a respective attention weighting for each word in the sentence, where the attention weightings are obtained by normalizing the attention contributions for all of the words in the sentence such that a total of the attention weightings in the attention vector is 1.

The text processing circuitry 28 may use the trained model to classify unseen text. The text processing circuitry 28 may use classifications 200 obtained using the trained model to perform any suitable task. For example, the text processing circuitry 28 may use the classifications 200 to perform a search task or an indexing task. The text processing circuitry 28 may use the classifications 200 as input for a further task. For example, in an embodiment in which the sentences are obtained from radiology reports having associated image data, the classifications 200 may be used to identify images on which a further process such as segmentation is to be performed.

The text processing circuitry 28 may use the attention overlay 202 to perform any suitable task. For example, the text processing circuitry 28 may use the attention overlay to identify keywords.

In the embodiment of FIG. 12, both real data and synthetic data are used to train the model. In other embodiments, synthesized data may be used without real data to train a model.

By using text data synthesis to obtain synthetic data, the model may be taught about rare cases or rare terms. The use of text data synthesis may teach the model about correct reasoning. For example, templates may be used to teach the model about sentence structures that are complex or difficult to interpret. Text data synthesis may increase the number of sentences on which the model is trained. Increasing the number of sentences available may be useful in a medical context in which a quantity of available training data may be limited.

Difficult examples may appear at test or application time as well as at learning time. It may be desirable to allow online learning during test or application as well as offline learning that occurs during training.

FIG. 13 is a flow chart illustrating in overview a method of training a model using expert input. The expert input may be obtained during training, during test or during application.

The training circuitry 26 receives a clinical text corpus 210. The clinical text corpus 210 is annotated with a set of label classifications. The training circuitry 26 trains a model 212 on the clinical text corpus 210. The trained model 212 is then applied to a target text 214 and outputs classifications 216, 218.

In the example shown, the target text includes the sentence ‘The lateral ventricles are minimally asymmetric as previously which may be congenital or secondary to the left basal ganglia haemorrhage’. Classification 216 comprises a classification of ‘congenital’ as positive. Classification 218 comprises a classification of ‘haemorrhage’ as positive.

The target text 214 and classifications 216, 218 are provided to one or more experts for an expert analysis 220. The experts review the text and classifications. The experts identify any misclassified sentences.

In the example shown, the classification 216 of ‘congenital’ as positive is incorrect. The expert identifies that the classification 216 of ‘congenital’ as positive is incorrect in the expert analysis 220.

In the present embodiment, the experts create a template 222 from each misclassified sentence. For example, an expert may create a template from a misclassified sentence by replacing each medical term in the misclassified sentence with corresponding placeholder text. The expert may use any suitable input method.

In other embodiments, the data synthesis circuitry 24 creates a template 222 automatically for each misclassified sentence. The automatically created template is then verified by the experts. For example, an expert may review the template to check that the template has been created correctly. The review may comprise checking that all medical terms have been correctly substituted with appropriate placeholders. The review may comprise checking that the classification of the template is correct.

The data synthesis circuitry 24 obtains a set of entities 226 to populate the templates 222. Entities 226 can be extracted from synonym dictionaries 228 (e.g. from UMLS)/and or the original clinical text 210.

The data synthesis circuitry creates a set of synthetic data 224 by filling the templates 222 with appropriate entities 226.

The training circuitry 26 retrains the deep learning model 212 using both the original data 210 and the synthetic data.

In the embodiment of FIG. 13, the retrained deep learning model 212 is applied to the target text 214. When using the retrained deep learning model 212, ‘congenital’ is correctly classified as uncertain. Experts can then see the improved results. Results are improved by the use of expert input to identify misclassified terms, and by generating templates for sentences in which misclassifications have occurred.

The learning that is performed by the model may be described as active learning. The model may be trained on new examples as they arise. A model trained with synthetic data generated via template may make a correct prediction.

Template creation may form one step in an offline or online active learning system. In the embodiment of FIG. 13, users provide feedback on which cases are misclassified.

An algorithm may be trained to derive the templates or additional synonyms required to automatically solve misclassified cases.

FIG. 14 is a flow chart illustrating in overview a method of automatic template inference in accordance with an embodiment.

In the method of FIG. 14, if the expert simply marks data which is wrongly classified and states what the correct classification should be, the training circuitry 26 can train a method to propose suitable templates to solve this. Sets of counterfactual templates may be proposed. A counterfactual template may be a template that is generated from an original sentence, but is altered to have a different classification from that of the original sentence. For example, if a template directly derived from an original sentence has a positive classification, a counterfactual template may be a template that is based

A deep learning model 212 is trained using original data 210 and synthetic data 224. The original data 210 may comprise an annotated clinical text corpus as described above. The synthetic data 224 comprises a set of synthetic sentences which may be generated using any suitable text synthesis method, for example any one or more of the text synthesis methods described above with reference to FIG. 5.

A misclassified example 230 is identified, for example using an active learning method as described above with reference to FIG. 13. In FIG. 14, the misclassified example comprises a text 232 which reads ‘No immediate threat from swelling’.

An expert identifies that a prediction made by the deep learning model 212 for the text 232 is incorrect. The deep learning model 212 has predicted a classification of negative for swelling. The true classification for swelling is positive.

The expert provides an input to identify the incorrect prediction, using any suitable input device 18 and input method. The text synthesis circuitry 24 receives the input from the expert indicating that the text 232 is a misclassified example 230.

The text synthesis circuitry 24 inputs the text 232, the incorrect classification and the correct classification into an algorithm for template generation 234. The algorithm for template generation 234 also receives a set of existing templates 236. For example, the set of existing templates may be templates 236 that were used to generate the synthetic data 224. The algorithm for template generation 234 receives outputs from the deep learning model 212.

The algorithm for template generation 234 is trained to generate a set of templates. In the embodiment of FIG. 14, the set of templates includes counterfactual templates.

A proposed set of templates 238 is derived from text 232, which was identified as a misclassified example 230.

The proposed set of templates 238 includes three templates 240, 242, 244.

The first template 240 comprises the text ‘No threat from [ENTITY]’. First template 240 is an example of a positive classification. A structure of the first template 24- corresponds to a structure of the text 232.

The second template 242 comprises the text ‘No threat of [ENTITY]’. Second template 242 is an example of a negative classification, since the text ‘No threat of [ENTITY]’ indicates that ENTITY is not present. Second template 242 is a counterfactual template, having a different classification to that of text 232.

The third template 244 comprises the text ‘No threat from possible [ENTITY]’. Third template 244 is an example of an uncertain classification, since the use of ‘possible’ indicates that the presence of ENTITY is uncertain. Third template 244 is a counterfactual template, having a different classification to that of text 232.

Templates that are generated by the algorithm for template generation 234 may be used to generate further synthetic data input for further training deep learning model 212.

FIG. 14 shows a plurality of losses that are used for training the algorithm for template generation 234 and the deep learning model 212. Losses include newly trained model classification performance, simplicity of the templates (short, minimally different to existing templates) and similarity between counterfactual templates.

A first loss 246 minimizes a distance between existing templates 236 and proposed templates 240, 242, 244. For example, the distance may be a BLEU score (Bilingual Evaluation Understudy Score). The first loss 246 is used to generate new templates that are minimally different to existing templates.

A second loss 248 minimizes a distance (for example, a BLEU score) between counterfactual templates. For example, distances between first template 240 and second template 242, between first template 240 and third template 244 and between second template 242 and third template 244 may be minimized. Use of the second loss 248 may result in a similarity between counterfactual templates.

A third loss 250 minimizes a length of templates. For example, lengths of first template 240, second template 242 and third template 244 may be minimized. The third loss 248 is used to generate new templates that are short. Use of the first loss 246 and the third loss 248 may result in simple templates.

A fourth loss 252 is a data classification cross-entropy loss which is used in training the deep learning model 212. The fourth loss 252 trains the deep learning model to produce correct classifications.

A fifth loss 254 is a template classification cross-entropy loss. This loss trains the deep learning model 212 to produce correct classifications for synthetic data.

The fourth loss 252 and fifth loss 254 may be used to improve newly trained model classification performance.

The deep learning model 212 and the algorithm for template generation 234 may be trained in an iterative manner. Outputs of the deep learning model 212 may be reviewed to identify misclassified examples which may be provided to the algorithm for template generation 234. Templates generated by the algorithm for template generation 234 may be used to generate further synthetic data to train the deep learning model 212. The algorithm for template generation 234 may be trained to produce short templates that are similar to existing templates.

Optionally, the method of FIG. 14 may include human acceptance/rejection of templates to check they are correct. For example, an expert may review templates 240, 242, 244 which are generated by the algorithm for template generation 234. The expert may determine whether the templates are appropriate English sentences. The expert may determine whether the classifications of the templates are correct. Experts may also specify for which entities a template is valid.

In further embodiments, templates are used as a way to do continual learning. Continual learning may comprise continuing to train an algorithm on new data when the apparatus 10 does not have access to old data on which the algorithm was previously trained. Old data on which the model was previously trained may be referred to as a historical dataset.

In some circumstances, access to training data may be limited by time and/or by location. For example, medical training data may only be available at a certain institution. A model may be initially trained at the institution at which the medical training data is available, but then used at a different institution. Alternatively, access to training data may only be granted for a limited period.

In a continual learning scenario, a deep learning model 212 may be trained to classify findings or impressions that it has not previously encountered. The training to classify the new findings or impressions may be performed at a different time and/or in a different location to an initial training.

Templates may be used as a method of remembering previous data without having that data available. Templates may act as a meta-representation of the key information in a historical dataset. The templates may then be used to synthesize similar examples later. The examples may be similar to examples included in the historical dataset. The use of templates to generate examples that are similar to a now-unavailable dataset may be used to tackle the problem of learning without forgetting.

Therefore, even if the original training data is not available to the model, synthetic data that is similar to the original training data may be generated using stored templates.

A set of templates may be referred to as a template bank. The template bank may act as a memory to continuously provide synthesized data that exhibits key patterns that are important for classification. The template bank may be used to add new tasks and distributions as observed in future populations, even with little or no extra annotation.

Templates may be used to learn new tasks. Templates may be used to easily generate data for new classes and new distributions that are encountered. New tasks and new classes may include unseen classes for which only expert knowledge is available. The expert knowledge may comprise, for example, synonyms and/or relationships to other label classes. Templates may be used to perform zero-shot learning.

The idea of template data synthesis may be leveraged to propose a combination of templates with expert knowledge to produce synthetic data following a controllable distribution. Automated template inference using machine learning is proposed. The automated template inference may be from misclassified examples and/or from an annotation protocol. A template text synthesis mechanism is proposed for better expert feedback during active learning for text AI. A template text synthesis mechanism is proposed for continual learning for text AI.

Synthetic data may be created on the fly rather than simply creating a dataset for training or evaluation. For example, if a misclassified sentence is identified by an expert after the original training is complete, further training may be performed using a template derived from the misclassified sentence.

In experiments using methods described above, radiographic findings and clinical impressions were predicted using a radiology report dataset. The dataset was annotated by a clinical researcher and two medical students. Experimental results were obtained for different synthetic datasets. It was found that results improved with more synthetic templates being added. A model trained on original data and simple templates was found to perform better than a model trained on original data alone. A model trained on original data, simple templates and permuted templates was found to perform better than a model trained on original data and simple templates. A model trained on original data, simple templates, permuted templates and combined templates was found to perform better than a model trained on original data, simple templates and permuted templates.

In an experiment, radiographic findings and clinical impressions were extracted from a dataset consisting of 27,000 radiology reports. The data was augmented with synthetic sentences ranging from simpler to expert-guided templates. A target dataset contained 27,000 radiology reports having a format similar to that shown in FIG. 4.

Training and validation test sets were as described in Schrempf, P.; Watson, H.; Mikhael, S.; Pajak, M.; Falis, M.; Lisowska, A.; Muir, K. W.; Harris-Birtill, D.; O'Neil, A. Q. Paying Per-Label Attention for Multi-label Extraction from Radiology Reports. Interpretable and Annotation-Efficient Learning for Medical Image Computing; Cardoso, J.; Van Nguyen, H.; Heller, N.; Henriques Abreu, P.; Isgum, I.; Silva, W.; Cruz, R.; Pereira Amorim, J.; Patel, V.; Roysam, B.; Zhou, K.; Jiang, S.; Le, N.; Luu, K.; Sznitman, R.; Cheplygina, V.; Mateus, D.; Trucco, E.; Abbasi, S., Eds.; Springer International Publishing: Cham, 2020; pp. 277-289.

Data was extended with an independent test set consisting of 317 reports, a prospective test set of 200 reports and an unlabelled test set of 27,000 reports. An exact number of patients, reports and sentences for each subset can be seen in Table 1.

TABLE 1

| Dataset | #patients | #reports | #sentences |
|---|---|---|---|
| Training | 138 | 138 | 839 |
| Validation | 92 | 92 | 515 |
| Independent test | 317 | 317 | 1,950 |
| Prospective Text | 197 | 200 | 1,411 |
| Unlabelled test | 10,113 | 29,399 | 249,025 |

An annotation process was performed in two phases. During the first annotation phase, the training, validation and independent test reports were manually split into sentences and subsequently labelled by a clinical researcher and two medical students. Each sentence was annotated independently. Sentences from the same original radiology report were allocated to the same data subset to avoid data leakage.

The second annotation phase was performed using the brat rapid annotation tool (Stenetorp, P.; Pyysalo, S.; Topic', G.; Ohta, T.; Ananiadou, S.; Tsujii, J. brat: a Web-based Tool for NLP-Assisted Text Annotation. Proceedings of the Demonstrations at the 13th Conference of the European Chapter of the Association for Computational Linguistics; Association for Computational Linguistics: 431 Avignon, France, 2012; pp. 102-107). Sentences were not manually extracted. Instead labels were assigned at word or phrase-level. A pipeline was extended to automatically extract sentences from this annotated data.

A list of 32 radiographic findings and clinical impressions found in stroke radiology reports was collated by a clinical researcher. This was the set of labels that the experiment aimed to classify. The set of labels was as shown in FIG. 6.

Each sentence was labelled for each finding or impression as “positive”, “uncertain”, “negative” or “not mentioned”. The most common labels such as “haemorrhage”, “infarct” and “hyperdensity” had between 200-400 mentions in the training dataset (100-200 negative, 0-50 uncertain, 100-200 positive). The rarest labels such as “abscess” or “cyst” only occurred once in the training dataset.

The training set was augmented by synthesising sentences for each label. Summary statistics for the synthetic datasets are shown in Table 2.

TABLE 2

| Dataset | #template sentences | #generated sentences |
|---|---|---|
| Random insertion | — | 840 |
| Random deletion | — | 840 |
| Simple templates | 3 | 99 |
| Permuted templates | 6 | 198 |
| Combined templates | 1 | 400 |
| Knowledge-injection | 3 | 1038 |
| templates | | |
| Protocol-derived | 4 | 400 |
| templates | | |

A larger number of synthetic sentences were generated for some templates than for others. When these synthetic datasets are then combined, the number of synthetic sentences is larger than the number of original sentences. It was observed that using a number of synthetic sentences that is larger than the number of original sentences had a negative effect on training. A sampling ratio between real and synthetic sentences was implemented to ensure that only 10% of samples in each batch are from the synthetic dataset. This was applied across all synthetic approaches, including baselines.

Baseline approaches, simple templates, permuted templates, combined templates, knowledge-injection templates and protocol-derived templates were as described above with reference to FIG. 5.

A plurality of models were compared. A set of labels is denoted as L and a set of certainty classes as C, such that the number of labels n_{L}=|L| and the number of certainty classes n_C=|C|. For all methods, data was pre-processed by extracting sentences and words using the NLTK library (Loper, E.; Bird, S. NLTK: The Natural Language Toolkit. In Proceedings of the ACL Workshop on Effective Tools and Methodologies for Teaching Natural Language Processing and Computational Linguistics. Philadelphia: Association for Computational Linguistics, 2002.), removing punctuation, and converting to lower case. Hyperparameter search was performed through manual tuning on the validation set, based on the micro-averaged F1 metric.

For neural network architectures, additional pre-processing was performed. Each input sentence was limited to nₜₒₖ tokens and padded with zeros to reach this length if the input is shorter. nₜₒₖ=50 was chosen as this is larger than the maximum number of words in any of the sentences in the dataset. The neural network models all finish with n_{L} softmax classifier outputs, each with n_{C} classes. The neural network models were trained using a weighted categorical cross entropy loss and Adam optimiser (Kingma, D. P.; Ba, J. Adam: A Method for Stochastic Optimization. 3rd International Conference on Learning Representations, ICLR, San Diego, Calif., USA, May 7-9, 2015, Conference Track Proceedings; Bengio, Y.; LeCun, Y., Eds., 2015.)

Weight was performed across the labels but not across classes. Given a parameter β which controls the power of the label weighting, the number of sentences n and the number of “not mentioned” occurrences of a label oₗ, weights were calculated for each label using the training data as follows:

$${w}_{l , {}^{″} not {mentioned}^{″}} = {( \frac{n}{{o}_{l}} )}^{β}$$

$${w}_{l , {}^{″} {mentioned}^{″}} = {( \frac{n}{n - {o}_{l}} )}^{β}$$

Models trained included Bag of Words+Random Forest, Word2Vec (Mikolov, T.; Sutskever, I.; Chen, K.; Corrado, G. S.; Dean, J. Distributed representations of words and phrases and their compositionality. Advances in neural information processing systems, 2013, pp. 3111-3119), BERT models (Devlin, J.; Chang, M. W.; Lee, K.; Toutanova, K. BERT: Pre-training of Deep Bidirectional Transformers for Language Understanding. Proceedings of the 2019 Conference of the North American Chapter of the Association for Computational Linguistics: Human Language Technologies, Volume 1 (Long and Short Papers); Association for Computational Linguistics: Minneapolis, Minn., 2019; pp. 4171-4186.doi:10.18653/v1/N19-1423; Alsentzer, E.; Murphy, J.; Boag, W.; Weng, W. H.; Jindi, D.; Naumann, T.; McDermott, M. Publicly Available Clinical BERT Embeddings. Proceedings of the 2nd Clinical Natural Language Processing Workshop; Association for Computational Linguistics: Minneapolis, Minn., USA, 2019; pp. 72-78. doi:10.18653/v1/W19-1909; Gu, Y.; Tinn, R.; Cheng, H.; Lucas, M.; Usuyama, N.; Liu, X.; Naumann, T.; Gao, J.; Poon, H. Domain-specific language model pretraining for biomedical natural language processing. arXiv preprint arXiv:2007.15779) and ALARM-based models (Wood, D.; Guilhem, E.; Montvila, A.; Varsaysky, T.; Kiik, M.; Siddiqui, J.; Kafiabadi, S.; Gadapa, N.; Busaidi, A. A.; Townend, M.; Patel, K.; Barker, G.; Ourselin, S.; Lynch, J.; Cole, J.; Booth, T. Automated Labelling using an Attention model for Radiology reports of MRI scans (ALARM). Medical Imaging with Deep Learning, 2020).

It was found that the use of templates improved performance on the independent test set. The difference was mainly noticeable in the rarer classes.

The best-performing trained model was compared to a rules-based system (Grivas, A.; Alex, B.; Grover, C.; Tobin, R.; Whiteley, W. Not a cute stroke: Analysis of Rule- and Neural Network-based Information Extraction Systems for Brain Radiology Reports. Proceedings of the 11^{th} International Workshop on Health Text Mining and Information Analysis; Association for Computational Linguistics: Online, 2020; pp. 24-37. doi:10.18653/v1/2020.louhi-1.4) and was found to have a higher accuracy than the rules-based system.

In general, rules-based models may be difficult to adapt to a slightly different task. In a typical rules-based model, rules must be re-written if the target labels change. By comparison, deep learning methods may be quite easily trained on a different dataset.

In embodiments described above, rules are introduced by an annotation protocol. Rules may be created in an annotation protocol to handle cases of ambiguous wording to ensure consistent interpretation. Labelling rules may be directly derived from a protocol.

Training data is augmented with synthetic data generated from templates. Methods described above may be very adaptable to new labels as templates may be used to generate new training examples.

Although the embodiments above are described with regard to medical data, in other embodiments any text data may be processed using methods described above.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments are described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical information processing apparatus for processing medical text data using a medical text processing model, the apparatus comprising processing circuitry configured to:
receive a portion of medical text data;
determine a template corresponding to the portion of medical text data, a classification label associated with the template, and a first medical term included in the portion of medical text data;
specify a second medical term which is different from the first medical term, wherein the second medical term is related to the first medical term; and
generate synthetic text data based on the second term by inserting the second medical term into the template.

2. The medical information processing apparatus according to claim 1, wherein the processing circuitry is further configured to train the medical text processing model using the synthetic text data and the classification label.

3. The medical information processing apparatus according to claim 1, wherein the synthetic text data is generated by inserting the second term into the template in a position corresponding to a position of the first medical term in the portion of medical text data.

4. The medical information processing apparatus according to claim 1, wherein the processing circuitry is further configured to:
determine at least one of the first medical term, the second medical term, the template, the label for each of a plurality of predetermined data distribution groups.

5. The medical information processing apparatus according to claim 1, wherein the second medical term is a synonym of the first medical term.

6. The medical information processing apparatus according to claim 5, wherein the processing circuitry is further configured to determine the synonym of the first medical term using at least one of: a database, a knowledge base, a knowledge graph, an ontology.

7. A medical information processing apparatus according to claim 1, wherein the portion of medical text data is a sentence or part of a sentence.

8. A medical information processing apparatus according to claim 1, wherein the classification label comprises a classification of the portion of medical text data as positive, negative or uncertain with respect to the first medical term.

9. A medical information processing apparatus according to claim 1, wherein the processing circuitry is configured to receive a classification of the portion of medical text data from an expert user and to determine the template and the classification label using the classification received from the expert user.

10. A medical information processing apparatus according to claim 1, wherein the template is verified by an expert user.

11. A medical information processing apparatus according to claim 10, wherein the processing circuitry is configured to receive from the expert user a set of medical terms for which the template is valid, and to specify the second medical term using the set of medical terms.

12. The medical information processing apparatus according to claim 1, wherein the portion of medical text data further comprises a third medical term having a relationship with the first medical term; and
the processing circuitry is further configured to specify a fourth medical term having a relationship with the second medical term, wherein the relationship between the second and fourth medical terms corresponds to the relationship between the first and third medical terms.

13. The medical information processing apparatus according to claim 12, wherein the first and third medical terms are findings, and the second and fourth medical terms are impressions.

14. The medical information processing apparatus according to claim 12, wherein the processing circuitry is further configured to receive a set of known relationships between medical terms, and to specify the second and fourth medical terms such that the relationship between the second and fourth medical terms is valid.

15. A medical information processing apparatus according to claim 1, wherein the determining of the template is in response to:
receiving, by the processing circuitry, a previous classification of the portion of medical image data that was obtained by processing the portion of medical image data using the medical text processing model; and
receiving, by the processing circuitry and from an expert user, an indication that the previous classification was incorrect.

16. A medical information processing apparatus according to claim 1, wherein the processing circuitry is further configured to determine at least one counterfactual template associated with a different, for example, opposite classification label to the classification label associated with the template, and to generate further synthetic text data using the at least one counterfactual template.

17. A medical information processing apparatus according to claim 1, wherein at least one of a) to c):—
a) the first medical term comprises an entity and the second medical term comprises a further entity;
b) the first medical term comprises a finding and the second medical term comprises a further finding;
c) the first medical term comprises an impression and the second medical term comprises a further impression.

18. A medical information processing apparatus according to claim 1, wherein the processing circuitry is further configured to store a plurality of further templates without storing corresponding portions of medical text data from which the further templates were derived, to use the stored plurality of templates to synthesize further text data, and to train the medical text processing model on the further synthesized text data.

19. A medical information processing apparatus according to claim 18, wherein the processing circuitry is configured to use the stored plurality of further templates to add new tasks and distributions to the medical text processing model.

20. A medical information processing apparatus according to claim 1, wherein the processing circuitry is further configured to create a combined template by combining the template with a further template, to synthesize text data using the combined template, and to train the medical text processing model using the text data that was synthesized using the combined template.

21. A medical information processing method comprising:
receiving a portion of medical text data;
determining a template corresponding to the portion of medical text data, a classification label associated with the template, and a first medical term included in the portion of medical text data;
specifying a second medical term which is different from the first medical term, wherein the second medical term is related to the first medical term; and
generating synthetic text data based on the second term by inserting the second medical term into the template.
